(19) United States
(12) Patent Application Publication
(10) Pub. No.: US 2006/0263452 A1
(43) Pub. Date: Nov. 23, 2006

(54) Topical composition containing essential oils

(76) Inventor: Alwyn Dowell, New Orleans, LA (US)

Correspondence Address: KEATY PROFESSIONAL LAW CORPORATION;THOMAS S. KEATY, 2 CANAL STREET, 2140 WORLD TRADE CENTER, NEW ORLEANS, LA, 70130, US

(21) Appl. No.: 11/130,899

(22) Filed: May 17, 2005

(51) Int. Cl. A61K 36/61; A61K 31/60; A61K 31/355; A61K 31/22; A61K 31/12; A61K 31/045

(52) U.S. Cl. 424/725.1; 424/742; 514/690; 514/159; 514/729; 514/546; 514/458

(57) ABSTRACT

A composition and method for treating and controlling a skin disorder associated with acne vulgaris, androgenetic alopecia, seborrheic dermatitis, atopic eczema, psoriasis, pseudofolliculitis barbae uses odorants in a pharmaceutically suitable carrier. The odorants can be essential oils, such as eucalyptol, camphor white oil, menthol, and thymol and also contain one or more phenols. The composition also has antioxidants A and E or derivatives thereof mixed with water.

### BACKGROUND OF THE INVENTION

The present invention relates to a composition for treating and controlling adverse effects of acne vulgaris, androgenetic alopecia, seborrheic dermatitis, atopic eczema, psoriasis, and pseudofolliculitis barbae.

The etiology and pathophysiology of most chronic skin, scalp and related disorders, such as male and female hair loss, seborrheic dermatitis (scalp psoriasis), atopic eczema (a long lasting, chronic skin disorder) and psoriasis, are not yet understood. Researches investigated such factors as low blood flow to specific areas of the skin, deficiency of nutrients and vitamins, microbially-driven inflammatory changes and the like. Some researches suggest that circulating hormones, more specifically dihydrotestosterone (DHT) is responsible for seborrheic dermatitis, which appears on sebum-rich areas of the scalp, face, and trunk, atopic eczema (atopic dermatitis), acne vulgaris, androgenetic alopecia (inherited hair loss) and psoriasis. Androgenic hormones promote growth of facial and body hair throughout life. In Early life, the growth of scalp hair also depends on androgenic hormones. As men age, androgenic hormones switch from promoting growth of scalp hair to promoting its loss, resulting in a condition known as androgenic effluvium and alopecia.

The androgenic hormones act through androgenic receptors, a cellular protein transcription factor which interacts with a specific region of DNA. Testosterone and its analog DHT must bind to androgenic receptors first to become active. Scalp androgenic hormones are derived either from the systemic circulation and/or synthesized in the skin and have been shown to bind to androgenic receptors located in the hair follicles.

The pathogenesis of pseudofolliculitis barbae is an inflammatory reaction to ingrown facial hair and ingrown hair along the nape of the neck. It is more common in people with curly hair and predominantly affects black males, who shave and receive a hair lining during a haircut. It was suggested that the only consistently effective treatment for pseudofolliculitis barbae is for the patient to grow a beard. Special razor have been used with varying results. A depilatory substance may be used every 2-3 days but is often irritating. Tretinoin (retinoic acid) 0.05% liquid to cream or 10% benzoyl peroxide cream may be effective in mild to moderate cases. Pseudofolliculitis barbae is al susceptible to pathogenic bacteria as well as fungal and viral infections exacerbating this skin disorder.

Several patents were issued directed to solving the problem of skin conditions mentioned above. For instance, U.S. Pat. No. 5,512,275 issued to Buck et al. relates to a method of using between 3% to about 20% by weight of spirits of camphor as a rubefacient.

U.S. Pat. No. 5,609,858 issued to Buck et al. relates to a method of treatment for androgenetic alopecia using topical application of Liquor Carbonis Detergens.

U.S. Pat. No. 5,629,002 issued to Weuffen et al. discloses a method of treating hair loss using skin-active vitamin Dexpanthenol.

U.S. Pat. No. 6,103,272 issued to Keeney et al. relates to a method of stimulating hair growth by administering colloidal silver.

U.S. Pat. No. 6,355,649 issued to Gormley et al. discloses a method of treating and/or reversing androgenetic alopecia and promoting hair growth, and methods of treating acne vulgaris, seborrhea and female hirsutism (male-pattern hair growth) by administering to the patient a 5.alpha.-reductase 2 inhibitor, such as finasteride, in a dosage amount under 5 mgs/day.

U.S. Pat. No. 6,768,026 issued to Mamana et al. discloses a method of treating conditions mediated by blocking non-essential androgen receptors in a patient. The composition comprises an effective dose of 3-(5-methoxyheptyl)-2-cyclohexenone.

U.S. Pat. No. 6,444,465 issued to Meisner et al. discloses the use of oligonucleotides in inhibiting the function of nucleic acid molecules encoding Her-1, ultimately modulating the amount of Her-1 produced. This is accomplished by providing oligonucleotides complementary to mRNA which specifically hybridize with mRNA or DNA encoding Her-1.

U.S. Pat. No. 6,503,953 issued to Vyden et al. discloses a method of treating an atopic disorder in a patient by administering an effective amount of at least one of an antifungal and an antibiotic over a period of time, while reducing the application of emollients to the patient by at least about 50%.

U.S. Pat. No. 6,703,009 issued to Rosen et al. relates to a topical composition and methods for treating pseudofolliculitis barbae and ingrown hair by applying a composition containing up to 18% of acetylsalicylic acid in a solvent containing up to 27% of isopropyl alcohol, ethanol, and optionally, methanol or water.

U.S. Pat. No. 6,843,983 issued to Bright-Ellington et al. discloses a shaving preparation used to prevent pseudofolliculitis barbae and ingrown hair; the disclosed substance contains glucocorticoid and an antibacterial agent. Optionally, the shaving preparation may also include a therapeutic amount of benzoyl peroxide.

While the above treatments may work satisfactory for the specific condition, there exists a need for a topical composition, which can be applied to the affected areas for controlling adverse effects of DHT on the human skin.

### SUMMARY OF THE INVENTION

It is, therefore, an object of the present invention to provide a composition and method of treating skin disorders, which are affected by the release of dihydrotestosterone.

It is another object of the present invention to provide a composition of matter and a method of treating skin disorders, such as for instance, acne vulgaris, androgenetic alopecia, seborrheic dermatitis, atopic eczema, psoriasis, and pseudofolliculitis barbae.

These and other objects of the invention are achieved through a provision of a topical compound, which comprises at least one odorant agent, such as an essential oil mixed with a pharmaceutically acceptable carrier and solvent. A preferred formulation of the topical agent of the invention generally comprises: from about 5.2% by total weight of one or more odorants mixed with up to about 94.8% of one or more of pharmaceutically acceptable carrier (alcohol), solvent (water), preservative, and vitamins A and E or salts, or esters thereof. The most preferred embodiment comprises eucalyptol, camphor white oil, coal tar solution in ethyl alcohol, menthol, methyl salicylate, thymol, vitamin E acetate, vitamin A palmitate, benzoic acid, and ethyl alcohol mixed with water.

### DETAILED DESCRIPTION OF THE PREFERRED EMBODIMENT

The topical composition of the instant invention is designed to treat and control the adverse effects of skin conditions, such as, without limitation acne vulgaris, androgenetic alopecia in men and women, seborrheic dermatitis, atopic eczema, psoriasis, pseudofolliculitis barbae and other such diseases. The composition of matter of the present invention may be used in cosmetic and/or pharmaceutical compositions, such as skin lotions, shampoos, face and body washes, hair conditioners, hair tonics, sun screen lotions, shaving creams, after-shaving lotions and other topical substances.

The composition of the present invention may be in the form of a solution, spray, lotion, cream, gel or ointment. The preferred form of the composition depends upon the condition being treated and the desired therapeutic effect. In general, the effectiveness of the composition is directly related to the form of the composition, with the ointment form being stronger than gels, gels being stronger than creams, creams being stronger than lotions and solutions.

The composition of matter may be used as a stand-along product or combined with other ingredients adapted for a particular desired application. It is envisioned that initially, the users will be encouraged to apply the product containing the composition of matter of the present invention once a day for five days, after which time the product could be used according to the personal hygiene schedule of the individual using the product. Once the adverse effects of the disorder are reversed, the product will continue controlling recurrence of the condition by regular application.

As an essential constituent in the concentrate compositions according to the present invention there are present one or more natural or botanical oils, sometimes also referred to as “essential oils.” By way of non-limiting example these include one or more of camphor oil, white, camphor powder synthetic technical, eucalyptol, eucalyptus oil, eucalyptus citriodora, menthol crystals, methyl cedryl ketone, methyl chavicol and methyl salicylate. The particularly preferred oils are eucalyptol, camphor white oil, menthol and methyl salicylate. These oils may be present in the composition in any amounts which are effective in providing a desirable skin repairing effect. Generally amounts from as little as 0.1% wt. to amounts of about 15% wt. are useful, based on the total weight of the concentrated liquid composition. More preferably these oils are present in amounts of from 0.1% wt., still more preferably from 4.8% wt. Of course, more than one oil may be used in a particular composition.

Eucalyptol, menthol, methyl salicylate and phenols, such as thymol, are odorants that may increase or decrease blood flow and/or the permeability of active ingredients through the skin of the patient. Menthol is often used as an anti-itch agent. Methyl salicylate may function here as an active drug agent, as a vasodilator compound and may also facilitate penetration of the compound through the patient's skin. It is preferred that the topical composition include at least one compound, which is an antiseptic and/or an anesthetic.

Each of these botanical oils is commercially available. The primary examples of such compounds: menthol, eucalyptol, and thymol are either obtained from natural sources such as naturally occurring oils or are derived from such oils. Eucalyptol is an essential oil and a terpene ether. Thymol may be derived from thyme oil or other oils. Menthol may be obtained from peppermint oil or other oils. The odorant agents are generally used in the compositions at levels of from about 0.4% to about 39%, by weight of the composition.

The composition of the instant invention is aqueous in nature. Water is added in order to provide 100% by weight of the concentrate composition. The water may be tap water, but is preferably distilled, purified and/or deionized water. If tap water is used, it is preferred that it is first filtered to remove organic and inorganic impurities, such as mineral salts.

The topical composition further comprises one or more antioxidants, including vitamin antioxidants, which inhibit oxidation or suppress reactions promoted by oxygen, oxygen free radicals (OFR), oxygen reactive species (ORS) including peroxides. The antioxidants useful in the present invention are preferably vitamin antioxidants that may be selected from the group consisting of all forms of Vitamin A including Vitamin A palmitate and 3,4-didehydroretinal, all forms of tocopherol such as Vitamin E (Alpha-tocopherol, 3,4-dihydro-2,5,7,8-tetramethyl-2-(4,8,12-trimethyltri-decyl)-2H-1-benzopyran-6-ol), .beta.-tocopherol, gamma-tocopherol, delta-tocopherol, tocoquinone, tocotrienol, and Vitamin E esters which readily undergo hydrolysis to Vitamin E such as Vitamin E acetate and Vitamin E succinate, and pharmaceutically acceptable Vitamin E salts such as Vitamin E phosphate, prodrugs of Vitamin A and Vitamin E, pharmaceutically acceptable salts of Vitamin A and Vitamin E, and mixtures thereof. Preferably, the antioxidant is selected from the group of lipid-soluble antioxidants consisting of Vitamin A palmitate, Vitamin E acetate and mixtures thereof.

Other conventional additives known to the art are also included in the compositions according to the instant invention. By way of non-limiting example and without limitation these include: a pharmaceutically acceptable carrier, a pharmaceutically acceptable solvent and a preservative. Exemplary preservatives, antioxidants, and chemical stabilizers include alcohol, such as for instance ethyl alcohol, and preservative, for instance an aromatic acid, such as benzoic acid USP. In the preferred embodiment, the composition of the present invention uses a hydrocarbon solvent. Examples of such hydrocarbon solvents include, but are not limited to, liquid mixtures of hydrocarbons distilled from petroleum, such as coal tar. Other solvents, such as polar solvents are used. The solvent that is used in the preferred embodiment is ethyl alcohol, which also facilitates delivery of the composition and serves as a permeation or penetration enhancer. Generally the total weight of such further additives may comprise up to 46% by weight of a concentrated composition formulation before water is added. Water may serve as a solvent and a carrier in combination with an alcohol.

A preferred formulation of the topical agent of the invention generally comprises: from about 3% by total weight of one or more odorants mixed with up to about 97% of one or more of alcohol, pharmaceutically acceptable carrier, solvent, preservative, vitamins and water.

Another preferred formulation of the topical agent of the invention generally comprises: an effective amount of each of the following components: eucalyptol, camphor white oil, coal tar solution, menthol, methyl salicylate, thymol, DL-a-tocopheryl acetate (vitamin E acetate), vitamin A palmitate, benzoic acid and ethyl alcohol admixed with purified water, and wherein the effective amounts by total weight are preferably about: 3%-15% eucalyptol, 1%-10% camphor white oil, 0.1%-5% coal tar solution, 0.4%-4% menthol, 0.4%-5% methyl salicylate, 0.4%-5% thymol, 1%-10% vitamin E acetate, 0.1%-3% vitamin A palmitate, 0.1%-1% benzoic acid, and 30-40% ethyl alcohol mixed with water.

The following example illustrates the compositions of the described invention. The exemplified compositions are illustrative only and do not limit the scope of the invention. Unless otherwise specified, the proportions in the examples and elsewhere in the specification are by weight.

### EXAMPLE

| Component | Amount (%) |
|---|---|
| 1,8-Cineloe: 1,8-expoxy-p-menthane | 3% |
| 1,7,7-Trimethbicyclo(2.21.)Heptan-2-one | 1% |
| 3-Hydroxy-1-methy-4-ispropyl benzene | 0.4% |
| methyl 2-hydroxbenzene | 0.4% |
| hexahydrothymol | 0.4% |
| coal tar in a pharmaceutically acceptable carrier | 0.1% |
| pharmaceutically acceptable carrier | 40.0% |
| pharmaceutically acceptable solvent | 53.5% |
| preservative C₇H₆O₂ | 0.1% |
| Vitamin A acetate | 1% |
| Vitamin E palmitate | 0.1% |

In this Example, pharmaceutically acceptable carrier comprises ethyl alcohol, water or a mixture of alcohol and water, and pharmaceutically acceptable solvent is purified water.

In one of the preferred embodiments, the topical composition comprises:

350 ml of eucalyptol diluted in equal amount of ethyl alcohol; for instance, 350 ml (about 11.06% by total weight) of eucalyptol is diluted in 350 ml 190-proof ethyl alcohol;

100 ml of camphor white oil diluted in ethyl alcohol; for instance 100 ml of camphor white oil (about 3.16% by total weight) diluted in 450 ml of 190-proof ethyl alcohol;

50 ml of coal tar solution (about 0.16% by total weight) obtained by diluting 7 ml-9.5 ml of coal tar diluted in 40.5 ml-43 ml 200-proof ethyl alcohol;

25 mg menthol crystals (about 0.79% by total weight) diluted in 50 ml of 190-proof ethyl alcohol;

30 ml of methyl salicylate (about 0.94% by total weight) diluted in 50 ml of 190-proof ethyl alcohol;

32 mg thymol (about 1.01% by total weight) diluted in 190-proof ethyl alcohol;

120 ml of Vitamin E acetate (about 3.8% by total weight);

10 ml of Vitamin A palmitate (about 0.32% by total weight);

10 mg benzoic acid (about 0.32% by total weight);

975.5 ml of ethyl alcohol (about 30.85% by total weight); and

1500 ml of purified water (about 47.59% by total weight)

A preferred method of the invention for treating or reducing skin disorders comprises the steps of: providing a topical agent comprising, an effective amount of one or more eucalyptol, camphor white oil, coal tar solution, menthol, methyl salicylate, thymol, vitamin E acetate, vitamin A palmitate, benzoic acid and ethyl alcohol admixed with purified water, applying at least one coat of said topical agent directly on skin over one or more areas of skin disorder for an effective amount of time.

The composition is prepared by using one or more of the odorants, such as eucalyptol, camphor white oil and coal tar solution to form a first group. Menthol, methyl salicylate and thymol solutions are added to the purified water to form a second group. The first group and the second group are then slowly mixed with agitation to form a third group. Vitamin E acetate, Vitamin A palmitate and benzoic acid are slowly added to the third group with agitation. The resultant solution is applied to the affected area at least once daily for five days. Then, the solution is applied on a generally regular basis according to the user's hygienic habits.

The foregoing descriptions comprise illustrative embodiments of the present inventions. The foregoing embodiments and the methods described herein may vary based on the ability, experience, and preference of those skilled in the art. Merely listing the steps of the method in a certain order does not necessarily constitute any limitation on the order of the steps of the method. Many changes and modifications can be made in the composition of matter according to the present invention. I, therefore, pray that my rights to the present invention be limited only by the scope of the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method for treating disordered tissue, comprising:
identifying disordered tissue that comprises skin disorder associated with acne vulgaris; and
applying to said disordered tissue a treatment composition for application to and penetration into said disordered tissue, wherein said treatment composition comprises at least one odorant agent in an alcohol-based carrier.

18. The method of claim 17, wherein said treatment composition comprises eucaluptol, camphor white oil, coal tar solution, menthol, methyl salicylate, thymol, Vitamin A palmitate, Vitamin E acetate, benzoic acid, and ethyl alcohol mixed with water.

19. The method of claim 17, wherein said treatment composition comprises between about 3% and about 15% eucalyptol, between about 1% and about 10% camphor white oil, between about 0.1% and about 5% coal tar solution, between about 0.4% and about 4% menthol, between about 0.4% and about 5% methyl salicylate, between about 0.4% and about 5% thymol, between about 1% and about 10% vitamin E acetate, between about 0.1% and about 3% vitamin A palmitate, between about 0.1% and about 1% benzoic acid, and between about 30% and about 40% ethyl alcohol mixed with water.

20. (canceled)

21. (canceled)

22. (canceled)

23. (canceled)

24. (canceled)

25. (canceled)
